(19) United States
(12) Patent Application Publication
(10) Pub. No.: US 2024/0108834 A1
(43) Pub. Date: Apr. 4, 2024

(54) MULTI-THERAPY SYSTEMS, METHODS AND APPARATUSES FOR THE ALLEVIATION OF SLEEP DISORDERED BREATHING

(71) Applicant: Halare, Inc., Sewickley, PA (US)

(72) Inventor: Anthony C. Warren, Wien (AT)

(21) Appl. No.: 18/275,539

(22) PCT Filed: Feb. 4, 2022
(86) PCT No.: PCT/US2022/015199

§ 371 Date: Aug. 2, 2023

Related U.S. Application Data
(60) Provisional application No. 63/147,347, filed on Feb. 9, 2021.

(51) Int. Cl. A61M 16/00; G16H 20/40; G16H 40/63

(52) U.S. Cl. CPC A61M 16/0066; A61M 16/0003; A61M 16/024; G16H 20/40; G16H 40/63; A61M 2205/3303; A61M 2205/3576; A61M 2230/005; A61M 2230/04; A61M 2230/20; A61M 2230/42

(57) ABSTRACT

A portable smart device including one or more sensors configured to detect physiological parameter values of the user during the breath training session and a processor coupled to the one or more sensors. The processor is configured to receive Mode II physiological parameter values from a positive airway pressure (PAP) device utilized by the user during PAP therapy, the Mode II physiological parameter values measured by the PAP device during the PAP therapy, develop and execute a breath training session based on the Mode II physiological parameter values, analyze, the physiological parameter values of the user detected by the one or more sensors during the breath training session to determine Mode I physiological parameter values. The Mode I physiological parameter values measured by the portable smart device during the breath training session, and transmit the physiological parameter values to the PAP device for adjustment of the PAP therapy.

### TECHNICAL FIELD

The present disclosure relates to systems, methods and apparatuses for aiding patients to alleviate symptoms of sleep disordered breathing, (SDB) including but not limited to sleep apnea and hypopnea.

### BACKGROUND

A significant proportion of Sleep Disordered Breathing (SDB) relates to a condition characterized by repeated episodes of hypopnea (underbreathing) and apnea (not breathing) during sleep resulting in reduction in blood oxygen saturation (SpO₂), arousal from sleep and sympathetic nervous system activation. Pauses may last 10-20 seconds or more and can occur 20 to 30 times or more an hour. The most common type of SDB affecting approximately 85% of patients is referred to as Obstructive Sleep Apnea (OSA) in which physical obstruction of the airways occurs due to sleep related loss of upper airway dilator muscle tone. A second type of SDB, commonly referred to as Central Nervous System Apnea (CNSA) is less common and occurs when the brain region that controls breathing fails to send signals to breathing muscles in a timely manner. It is now generally believed that CNSA commonly occurs in combination with OSA and that the historical separation of OSA and CNSA insufficiently categorizes the disease forms.

### SUMMARY

An example embodiment includes a portable smart device designed to be held by a user or worn by the user, the portable smart device for determining a breathing quality of a user during a breath training session, the portable smart device comprising one or more sensors configured to detect physiological parameter values of the user during the breath training session and a processor coupled to the one or more sensors. The processor is configured to receive Mode II physiological parameter values from a positive airway pressure (PAP) device utilized by the user during PAP therapy, the Mode II physiological parameter values measured by the PAP device during the PAP therapy, develop and execute a breath training session based on the Mode II physiological parameter values, analyze, the physiological parameter values of the user detected by the one or more sensors during the breath training session to determine Mode I physiological parameter values, the Mode I physiological parameter values measured by the portable smart device during the breath training session, and transmit the physiological parameter values to the PAP device for adjustment of the PAP therapy.

An example embodiment includes a positive airway pressure (PAP) device having a mask worn by the user, the PAP device for monitoring apnea events during PAP therapy, the PAP device comprising a fan for applying positive airway pressure to the mask worn by the user during the PAP therapy, one or more sensors configured to detect physiological parameter values of the user during PAP therapy, and a processor coupled to the one or more sensors. The processor is configured to receive Mode I physiological parameter values from a portable smart device, the Mode I physiological parameter values measured by the portable smart device during a breath training session, develop and execute PAP therapy based on the Mode I physiological parameter values, analyze, the physiological parameter values of the user detected by the one or more sensors during the PAP therapy to determine Mode II physiological parameter values, and transmit the Mode II physiological parameter values to the portable smart device for adjustment of the breath training.

An example embodiment includes an integrated positive airway pressure (PAP) device having a mask worn by the user, the PAP device for monitoring apnea events during PAP therapy, and for performing breath training, the PAP device comprising a fan for applying positive airway pressure to the mask worn by the user during the PAP therapy, one or more sensors configured to detect physiological parameter values of the user during PAP therapy, and a processor coupled to the one or more sensors. The processor configured to detect, via the one or more sensors, Mode I physiological parameter values measured by the PAP device during a breath training session, develop and execute PAP therapy based on the Mode I physiological parameter values, analyze, the physiological parameter values of the user detected by the one or more sensors during the PAP therapy to determine Mode II physiological parameter values, adjust the breath training breath training based on the Mode II physiological parameter values, and adjust the PAP therapy based on the Mode I physiological parameter values.

### BRIEF DESCRIPTION OF DRAWINGS

So that the way the above-recited features of the present disclosure can be understood in detail, a more particular description of the disclosure, briefly summarized above, may be made by reference to example embodiments, some of which are illustrated in the appended drawings. It is to be noted, however, that the appended drawings illustrate only example embodiments of this disclosure and are therefore not to be considered limiting of its scope, for the disclosure may admit to other equally effective example embodiments.

FIG. 1 shows examples of apparatuses for implementing Mode I, according to an aspect of the disclosure.

FIG. 2 shows an example of a mask for implementing Mode II, according to an aspect of the disclosure.

FIG. 3 shows an example of a mask for implementing Mode I and Mode II combined, according to an aspect of the disclosure.

FIG. 4 shows an example of ear buds with sensors, according to an aspect of the disclosure.

FIG. 5 shows a system configuration for separate devices implementing Mode I and Mode II, according to an aspect of the disclosure.

FIG. 6 shows a system configuration for a single device implementing Mode I and Mode II, according to an aspect of the disclosure.

FIG. 7 shows an example of a flow diagram of a breath training session to aid SDB, according to an aspect of the disclosure.

FIG. 8 shows example hardware for the Mode I and Mode II apparatuses shown in FIGS. 1-4, according to an aspect of the disclosure.

### DETAILED DESCRIPTION

Various example embodiments of the present disclosure will now be described in detail with reference to the drawings. It should be noted that the relative arrangement of the components and steps, the numerical expressions, and the numerical values set forth in these example embodiments do not limit the scope of the present disclosure unless it is specifically stated otherwise. The following description of at least one example embodiment is merely illustrative in nature and is in no way intended to limit the disclosure, its application, or its uses. Techniques, methods, and apparatus as known by one of ordinary skill in the relevant art may not be discussed in detail but are intended to be part of the specification where appropriate. In all the examples illustrated and discussed herein, any specific values should be interpreted to be illustrative and non-limiting. Thus, other example embodiments may have different values. Notice that similar reference numerals and letters refer to similar items in the following figures, and thus once an item is defined in one figure, it is possible that it need not be further discussed for the following figures. Below, the example embodiments will be described with reference to the accompanying figures

This disclosure relates to systems, methods, and apparatuses for aiding patients suffering from sleep disordered breathing (SDB) including sleep apnea and hypopnea, by intimately combining two distinct treatment modes each dealing with different causes of SDB, Mode I for neuro-physical causes of SDB, and Mode II for physical causes of SDB. In general, SDB can have major short-term and long-term deleterious impacts. When sleep is interrupted throughout the night, drowsiness occurs during the day. People with SDB have twice the risk for car accidents, are 25% more likely to have at-work accidents and exhibit loss of work efficiency. SDB may also lead to long-term serious, chronic health issues, such as increased chance of stroke and other cardio-vascular diseases, and dementia. Roughly 38,000 cardiovascular deaths annually are in some way related to SDB.

SDB may be un-diagnosed in many cases until symptoms have become life threatening. Estimates are between twelve and twenty million Americans alone suffer from SDB. The economic impact of SDB in the US alone is estimated to be several billion dollars annually, which does not take into account the cost of long-term care associated with other chronic diseases triggered by SDB.

There are a variety of treatments for SDB. These include lifestyle changes, special pillows or devices to avoid sleeping on the back, and oral appliances to keep the airways open. If these are inadequate, continuous positive airway pressure devices (CPAP), the most prevalent type of Positive Airway Pressure devices (PAP). A PAP device uses a fan to blow air into the user's nose and/or mouth to provide the continuous positive airway pressure during PAP therapy. Due to discomfort, associated noise, and impact on personal lives, usage compliance of PAP is poor. Compliance rates range from 4.7 to 5.3 hr. per night. PAP generally needs to be used as long as there are symptoms.

Expiratory Positive Airway Pressure uses inserts in the nostrils worn during sleep. The inserts must have a tight seal to the nostril opening, and if the modes reverts to oral expiration, common with apnea patients, these inserts have little or no effect. As with PAP means, treatment must continue as long as symptoms occur.

Exercising tongue muscles has shown some benefit in maintaining upper airway clearance at night. A mouth insert device applies electrical currents to the tongue has shown some benefit for mild apnea cases. However, such a device does not address underlying neuro-physiological factors.

Surgical procedures are used to remove tissue from the airway. Success rates are about 65% and falling to less than 50% over time. Surgery is risky, costly and uncomfortable; most patients with moderate or severe SDB often still require PAP after surgery.

Implanted devices can detect an interruption in breathing and then stimulate nerves controlling the muscles associated with breathing. These methods are costly and are typically used in chronic situations where other treatments have failed. The solutions described herein overcome the problems in such existing methods for the alleviation of SDB by intimately combining two different, yet complementary modes of treatment.

Such an improved treatment of SDB arises from combining both physical and neurophysiological interventions to provide both a greater reduction of apnea symptoms and a longer-term reduction in therapeutic needs. In the systems described herein, neuro-physical interventions are the principal purpose of Mode I methods, and physical interventions are the principal purpose of Mode II methods.

Specifically, there is a need for a means to alleviate the symptoms of SDB caused by neuro-physiological instabilities that does not require surgical intervention, augments PAP machines and methods, can be employed safely and reliably in non-clinical locations such as the home and may reduce the need for long-term therapy. Retraining the autonomous breathing control centers in the brain offers such a means. Breathing is controlled by complex automatic processes involving the integration of neural signals originating at respiratory control centers in the brain. These centers generally exert tight control over respiratory rate, volume and breathing patterns. However, the motor cortex can override these involuntary mechanisms while humans are conscious. For example, regular “background” breathing is volitionally interrupted during speaking, or when swimming, such “interrupt commands” being initiated within the motor cortex. An individual can volitionally decide when to hold breath, when to breathe more shallowly or faster for example. During sleep, breathing is an autonomous function controlled within the pons and medulla oblongata, primitive sections of the brain which reside at the top of the spinal column in the brain stem. However, there is an interaction between these two centers allowing them to operate in tandem to control breathing. Moreover, this interaction enables neuro-plastic modification of the autonomous breathing patterns using volitional techniques.

Volitional breathing modulation provides a mechanism for humans to re-program autonomous breathing cycles. Training for a limited period can have lasting effects due to such neuro-plastic modifications of the brain control centers through so-called LongTerm Facilitation (LTF). This suggests that dysfunctional breathing patterns such as exist in SDB may be amenable to breathing retraining over a short period yet having long term benefits.

Dysfunctional breathing control with a tendency to breathing instability and hyperventilation have been observed in individuals with SDB during normal waking state. Increased tendency to hyperventilate as evidenced by heightened central chemosensitivity is a common finding in SDB where the extent of this increased chemosensitivity is positively related to the severity of OSA. Respiratory instability contributes to collapsibility of the airways in OSA. However, exposing subjects to daily sustained periods of hypercapnia can reduce the number of apnea events during sleep.

Further, patients diagnosed with OSA are more likely to exhibit unstable breathing in the wakened state. This is caused, in many cases, by the breathing control centers in the brain having a so-called heightened loop gain. A high loop gain can result in breathing patterns exhibiting periods of over and under breathing, similar to those typical of those observed in sleep apnea. Loop gain is a term generally used to describe the behavior of any system that has feedback. A well-known example is the howl created in a sound system when an input microphone picks up sound from loudspeakers. The sound then in turn gets amplified further. In any such system when the loop gain exceeds a value of 1, the system becomes unstable. Another example of loop gain is in the spread of a disease when an infected individual spreads a virus to more than one additional person whereby the disease spreads out of control. In a breathing control system, like the one described herein, if the breathing level drops drastically for some reason, such as due to a collapse of the upper airway during an apnea event, this reduces the oxygen concentration in the blood. If the body/brain over-corrects rather than settling to a stable state, gasping and over-breathing occurs, triggering yet another oxygen starvation event. Generally, if the breathing loop gain is greater than a value of 1, the breathing cycle and oxygen level oscillate resulting in symptoms observed in OSA.

In addition, observed high loop gain measured prior to the application of PAP therapies, is not reduced even after many weeks of PAP usage to mitigate sleep apnea. Thus, in general, PAP treatments do not produce a reduction in one of the major causes of sleep apnea. Cessation or partial usage of a PAP device leads to recurrence of SDB. A PAP type device manages the symptoms of OSA but does not affect a long-term cure for SDB, including CNSA.

Further, if a patient of OSA undertakes breathing exercises which may include application of minimal intermittent hypoxia cycling, the need of a PAP device may be reduced, for example, by lowering the pressure required to maintain upper airways open or reducing the time that pressure is applied.

In general, sleep apnea is defined into two distinct classes, OSA and CNSA. However, there are two underlying and inter-related causes even in OSA; physical features exhibited by the muscles and tissues in the upper airways that can reduce or fully restrict air-flow during sleep, and neuro-physiological causes that result in failure of neurological triggers to maintain regular breathing and to open up the upper air-ways through stimulation of the pharyngeal muscles. The former problem can be alleviated using PAP devices, the latter using appropriate breath training. Such breath training may have longer term benefits as it can result in neuroplastic modifications in the breathing control centers in the brain reducing the loop gain.

Patients exhibiting mild to moderate symptoms of OSA subjected to mild periodic hypercapnia to simulate apnea events while awake for short periods over a ten-day period, significantly reduced their apnea symptoms.

It is therefore possible for a patient with SDB to simulate such apnea events volitionally by retraining their breathing patterns while awake using the volitional breathing control brain center within the brain motor cortex.

Mode I is breath training during wakefulness aimed at reducing breathing instabilities triggered by malfunctions in breathing control centers in the brain.

Mode II is the use of Positive Airway Pressure (PAP) during sleep aimed at physically maintaining upper airway patency during sleep.

Each mode is aided by an apparatus that is able to sense and store relevant physiological and performance data. Multiple apparatuses can be dedicated to a respective one of the modes. Alternatively, a single apparatus may be used to sense and control both modes. Regardless of whether two physically separate apparatuses are used or a single apparatus is used, data is shared between the electronic means controlling each mode thereby improving the outcomes of one or both of the modes.

Specifically, in Mode I, the SDB patient, while awake, is guided through breathing exercises and monitored for compliance to a prescribed breath training regimen, and outcomes also measured and recorded by an apparatus.

In Mode II, the patient wears a mask that can provide positive airway pressure during the sleeping state to alleviate SDB, while data recording the length, timing and amplitude of Positive Airway Pressure interventions for apnea and hypopnea events are sensed and stored, as well as physiological parameters characterizing such events

The progress of the patient using Mode I over an extended time, may be used to modify the programs controlling Mode II functions and determining the level of usage that is required of the PAP apparatus. Similarly, the progress of the patient using Mode II over an extended time, may be used to modify the programs controlling Mode I functions and determining the extent and type of breath training that is required. Over time, the patient may be able to reduce the use of, or even discontinue, the need to use the PAP functions of an apparatus during sleeping and/or reduce or discontinue breath training while in an awakened state. Additionally, data recorded in Mode II, may be used to determine the appropriate breath training regimens in Mode I, such regimens being adjusted in the electronic controller for Mode I.

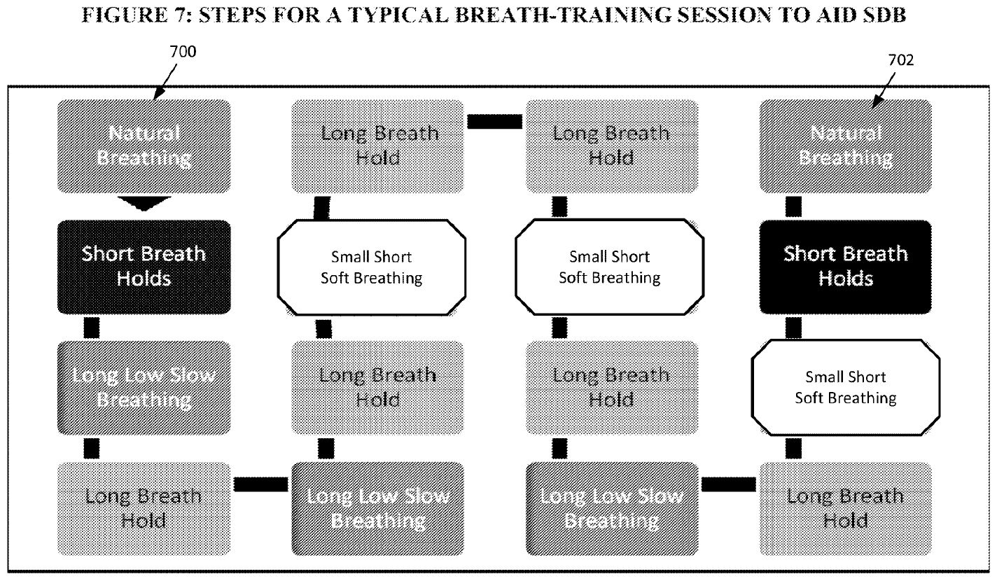

In one example of Mode I, patients improve their breathing control by undertaking a defined breath training regimen while awake which is electronically stored in a portable apparatus or uploaded securely to a central database, that guides a patient through breathing techniques and a prescribed breath-training regimen. A regimen is defined by a pattern of timed breathing sequences including different types of breath holds, slow deep breathing, shallow breathing, long exhales and other breathing activities. One such sequence is shown in FIG. 7 where the user starts with natural breathing at step 700 and proceeds through various steps of short breath holds, long breath holds, and soft breathing and finally finishing with natural breathing at step 702. Exercises typically take 10-30 minutes. Many different such breath training regimens are stored in a data base to be delivered appropriately to a patient depending on need. These may also be stored either permanently or temporarily in an apparatus programmed for Mode I.

While a breath training regimen is performed by a patient, the apparatus monitors and stores data which can be analyzed locally and/or centrally to determine adherence to the regimen and breathing performance trends over time.

Specifically, the breath training methods for Mode I are described in detail elsewhere including in U.S. Pat. No. 9,830,832 which is incorporated by reference herein. For example, automated systems, methods, and apparatuses for breath training can be undertaken at home. These systems and apparatus include a sensing device, with one or more sensors configured to detect physiological data (i.e. physiological parameter values) from a person during breath training. In these systems, a processor is coupled to the device and the sensor(s). The processor provides instructions to the trainee through the output device based on a personalized breath training regimen and is configured to receive and analyze the physiological data detected from the sensor(s). The breath training methods performed by these systems may include the steps of instructing a trainee based on a breath training regimen, detecting physiological data from the trainee through at least one sensor, analyzing the physiological data, and providing feedback to the trainee based on the analyzed physiological data. In one example, hyperventilation-based breath training requires that the subject is trained to manage hyperventilation cycles by voluntarily undertaking breath holding and reduced breathing levels referred to as self-imposed apnea and hypopnea events. The system may instruct the trainee to perform various breathing actions (e.g., natural breathing, short breath hold, long breath hold, light breathing, etc.) over a period of time during a training session via audio and/or video prompts, during which physiological data is monitored. The physiological data may then be analyzed to determine the efficacy of the breath training session and to generate an updated breath training session to improve the efficacy of the breath training session. Efficacy of the breath training session may be measured by indications of one or more of decreased heart rate, increased heart rate variability, reduced rest breathing rate, increased blood oxygen saturation levels, and increased breath holding time during the breath training session.

In general, an apparatus operating in Mode I monitors, collects and stores data on the patient's physiological signs including at least one or more of breathing patterns, heart-rate, heart-rate variability, breathing rate, vagal tone, blood oxygen saturation levels and breath holding times while the breathing exercises are undertaken. This are referred to as Mode I data.

In one example of Mode II, a second apparatus applies positive airway pressure (PAP) from a wearable mask while the patient is asleep to correct intervals of reduced or interrupted breathing. Mode II techniques are generally referred to as positive airway pressure methods (PAP). While a patient is using a mask that provides PAP, the apparatus monitors and stores and/or loads data to a central database which can be analyzed to determine adherence to the regimen and sleep pattern trends over time.

There are a number of different positive airway pressure (PAP) techniques including, but not limited to, Continuous Positive Airway Pressure (CPAP), BiLevel Positive Airway Pressure (BiPAP) and Expiratory Positive Airway Pressure (EPAP), which are considered to be categories of PAP for the purpose of this document.

Data capable CPAP machines are now available for OSA treatment therapy. Such a machine has software that allows a patient and/or caregiver to track the progress and performance of the therapy. The software is capable of collecting data to determine the efficiency of changes in the therapy. The gathered information is helpful in determining whether changes are needed in the current therapy.

In general, a data-capable apparatus operating in Mode II supplying positive airway pressure during sleep, monitors, collects and stores data related to one or more of compliance, therapy efficiencies, applied pressures, leakages, breathing interruption events, snoring episodes and other physiological factors such as Oxygen Saturation, (SpO₂), heartrate, heart rate variability and others.

Further standard indices that quantify the seriousness of SDB are derived. One such index is the Apnea-Hypopnea Index, (AHI) is the number of apneas or
hypopneas recorded per hour of sleep. It is generally expressed as the number of SDB events per hour. Based on the AHI, the severity of OSA may for example be classified as follows:
None/Minimal: AHI≤5 per hour
Mild: AHI≥5, but <15 per hour
Moderate: AHI≥15, but <30 per hour
Severe: AHI≥30 per hour

A second index may also be calculated from detected reductions in blood oxygen levels (desaturation) which often occur during SDB events. At sea level, a normal blood oxygen level (saturation) is 96-97%. Reductions to not less than 90% usually are considered mild. Dips into the 80-89% range can be considered moderate, and those below 80% are severe. The oxygen desaturation index (ODI) is commonly used to evaluate the severity of sleep apnea. ODI is defined as the number of episodes of oxygen desaturation per hour of sleep, with oxygen desaturation defined as a decrease in blood oxygen saturation (SpO₂) to lower than 3% below baseline. Based on the ODI, the severity of OSA may for example be classified as follows:
None/Minimal: ODI<5% decrease over defined time
Mild: ODI≥5, but <15% decrease over defined time
Moderate: ODI≥15, but <30% decrease over defined time
Severe: ODI≥30% decrease over defined time.

Data and index types such as AHI and ODI described in the previous three paragraphs (as well as other index types) are measured during PAP therapy and referred to as Mode II data. There are significant benefits of combining at least two therapies for the reduction of apnea symptoms in one apparatus or in two apparatuses that share data. PAP methods are well known and effective in many cases for managing physically induced symptoms. However, the masks and associated pumping device used while sleeping have several major disadvantages including but not limited to, noise, discomfort and impact on partner relationships. These disadvantages lead to low PAP compliance. In turn this can result in other chronic life-threatening co-morbidities. Moreover, PAP methods treat the symptoms rather than providing lasting relief and have limited, if any, impact on neuro-physiological triggers.

Breath training does not suffer from these disadvantages, but rather offers long-lasting reduction of symptoms after only a few weeks of daily exercises. If breath training does not provide a complete loss of symptoms, a combination with PAP therapy may be needed.

Combining these two modes into one or more apparatuses to support the two modes in tandem can improve therapeutic outcomes. Other benefits include reduction of patients' long-term dependence on the PAP methods, reduction of the requirements of the PAP device, and lessening compliance concerns. Patients may be able to abandon use of the PAP Mode II completely after a few weeks of breath training, or at least reduce their usage of and dependence upon a PAP device.

A user of the combined Mode apparatus(es) may commence their therapy using the conventional PAP methods and data collection, thus preserving established and proven methods and safety. The user then commences personalized regular breath training sessions during waking times. The PAP function monitors the apnea and hypopnea breathing interruptions and thereby the progress and effectiveness of the breath training can be recorded, analyzed and reported to an expert Health Care Provider (HCP) and/or the user. The breath training regimens can be based wholly or partly on the Mode II data. As breath training alleviates the SDB symptoms, the patient may be provided automatically, or by and expert, updated breath training regimens, based in full or in part, on the mode II data. In addition, stored programs controlling the PAP function can also be modified as the patient's symptoms are alleviated from breath training.

Over time, for example, undertaking breathing training for 12-20 minutes daily for six to twelve weeks, it is possible for the patient to reduce the use of the PAP function as well as the regular breath training until there may only be occasional therapeutic interventions required, or none.

The internet is increasingly being used to monitor critical care at home and to guide and monitor compliance of the mode(s) to therapeutic protocols, thereby increasing the reliability of treatment and improvement of outcomes. Connecting patients to an electronic data network offers additional advantages. For example, data from a population can be accumulated and analyzed to improve training methods that can benefit individual and groups of patients using co-called collaborative filtering algorithms. Further, patients that are made aware of their progress in comparison with a group of patients with similar aims are more likely to be more compliant to a training or therapeutic regimen and persevere resulting in better outcomes. The system/method as described herein advances tele-health practices for the alleviation of SDB symptoms using breath training in combination with PAP functions to overcome limitations of PAP functions alone as the internet can facilitate data exchange between two apparatuses or between one or more apparatuses and a central serving computer.

Combining both breath training means to be used when awake (Mode I) and PAP functions when asleep (Mode II) in one apparatus or in two apparatuses that can exchange data enables patients to gradually reduce their dependence on the PAP function safely. Further benefits can accrue when such apparatuses are components in a system. Such a system includes one or more of the following features: adaptability to different breath training regimens and individual patient's requirements; monitoring of patients' compliance to the prescribed regimen and identification of patients; monitoring of certain physiological data during breath training and sleep states to provide warnings if the data exceed defined limits; aggregating patients' data in a central database; analyzing this database for individual trends, benchmarking, regimen improvements, and peer-peer comparisons; providing feedback to patients from such analyses, using data collected while one or both modes are being undertaken to modify the programs for one or both of the modes.

As mentioned above, this disclosure relates to systems, methods, and apparatuses for alleviating the symptoms of sleep disordered breathing (SDB). Specifically, the patient is provided two apparatuses, each of which has a different Mode of operation. Alternatively, the patient is provided with one apparatus which can operate in two modes. One Mode, referred to herein as Mode I, guides a patient, while in a waking state, through one or more breath-training regimens while monitoring, and storing one or more physiological data referred to generally herein as Mode I Data. No expert is required to be present during the breath training sessions. These data can be stored within the apparatus used by the patient, such data being uploaded to a central database over a data transmitting network or using a portable data storage means, from time to time, whereupon they are analyzed to determine the progress or otherwise of the patient, to provide feedback on such progress or otherwise, to provide guidance on improving the patient's techniques. A second Mode, referred to herein as Mode II, is used while the patient is in a sleep state. The apparatus applies positive airway pressure (PAP) continuously or from time to time as needed to alleviate apnea and hypopnea events. In Mode II, the apparatus measures and stores certain physiological data such as heart-rate and heart-rate variability, breathing patterns and other data. These data are referred to herein generally as Mode II Data. No expert is required to be present during the PAP Mode. The data can be stored within the apparatus which is worn by the patient, such data being uploaded to a central database over a data transmitting network or using a portable data storage means, from time to time, whereupon they are analyzed to determine the progress or otherwise of the patient, to provide feedback on such progress or otherwise, to provide guidance on improving the patient's techniques, compliance requirements etc.

Data collected in one or both modes may be shared by software programs controlling and monitoring one or both modes such that the regimens of the modes can be modified based on data collected in one or both modes. As one example of the value of sharing data between modes, if it is recorded in Mode II that the symptoms of SDB are declining in either intensity, or frequency over time, the breath training Mode I may be modified to a more or less stringent exercise program. In addition, the hours needed for wearing the apparatus for Mode II operation may be reduced, or the applied positive pressure patterns modified.

Furthermore, Mode I Data indicating that the patient has improved their autonomous control over apnea and hypopnea events may be used to modify the patterns of positive airway pressure in magnitude, duration, and times thereby allowing the patient to become less dependent on PAP modes. The patient may be able to stop using a PAP mask at night, which would be a significant benefit to patients.

The data stored in the apparatus(es) from one or both Mode operations, may be transmitted and stored in such a way that the anonymity and personal data of the patient and/or an associated breathing expert may be maintained at least to the guidelines established under the US HIPAA specifications. In addition, data from a multiplicity of patients is analyzed in order to improve recommendations to an individual patient based on the accumulated data from the multiplicity. Data from a multiplicity of patients can also be used to determine improvements to breath-training and/or PAP regimens over time so that the outputs from breath training and PAP applications improve over time. The mining of data from a number of individuals in order to improve recommendations to members of a population is known as “collaborative filtering” and has, to date, not been applied to breath training, and PAP applications.

A central serving computer stores a library of breath-training regimens for use in Mode I. In addition, new regimens may be entered into the system by experts to be used by their specific patients or, by agreement, made available to patients. The breath-training regimens are improved as data acquired from patients during training are stored and analyzed using collaborative filtering algorithms on one or more central serving computers.

Experts may access the stored breath training regimens and share results with other experts when specifically permitted. Thus physicians or other healthcare professionals, pulmonologists and breath training experts can benefit from sharing knowledge and aggregated patient data on different regimens while preserving their and their patients' anonymity and personal data.

The following examples serve to illustrate applications of the system/method to alleviate the symptoms of SDB's using a combination of breath training and a PAP device.

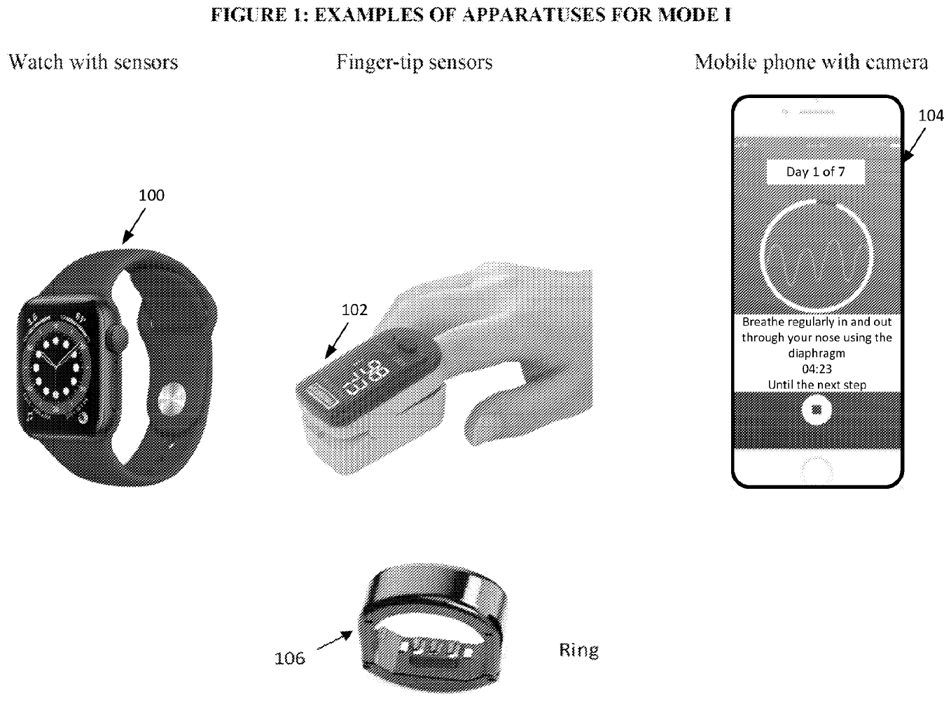

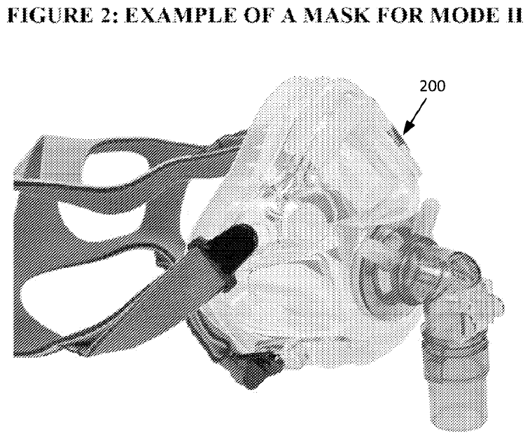

In a first embodiment, an apparatus designed for operating in Mode 1 uses a sensing device such as a wrist band sensor 100, a finger-tip sensor 102 or a camera embedded within a smart phone 104, or a ring 106 as shown in FIG. 1, or other means. Such sensors transmit sensed data to a computing device either wirelessly or hard-wired. Further, means to communicate directly to the user either or both visually and aurally are provided. Examples of aural devices are mask 200 shown in FIG. 2. One example of Mode I is the breathesimple application available on the Apple Appstore. The computing device has a means to connect to a data transmitting network from time to time, or to store data on a portable data storage device which can be used conveniently to transfer data to a data network, or a second device. Such a second device to provide Mode II functionality, such as shown schematically in FIG. 2, is able to apply PAP to alleviate the symptoms of SDB caused by physical restrictions in the upper airways.

The computing device connects continuously or from time to time to a secure data network with a central dedicated database. The database updates and stores at least modes' records, breath training courses, and PAP management instructions. In addition, the database may store data on phenotypes that may be used to match an individual user to similar users in order to provide personalization of breath training courses and PAP protocols. Modes' records including breathing health and sleep disturbance data acquired from the computing device used for Mode I and from the PAP Mode II device are stored with individual modes records.

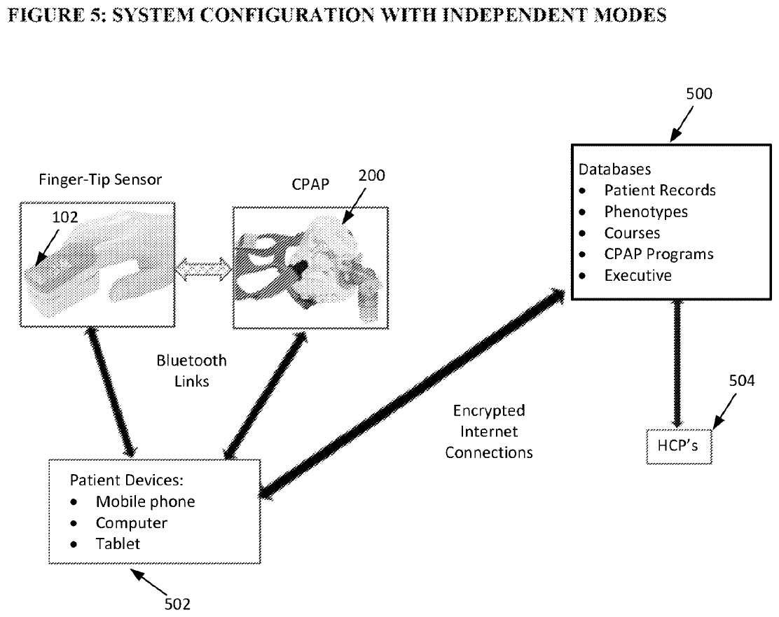

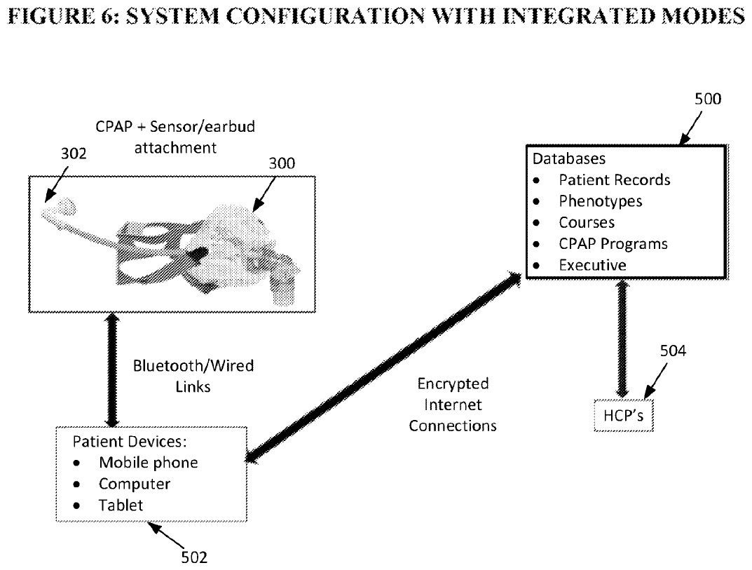

FIGS. 5 and 6 show the system configurations for Mode I and Mode II. In FIG. 5, a sensor device 102, such as a finger-tip sensor monitors physiological parameters during a guided breath training session. The data are analyzed by a local or centrally located computing device(s) 502. Specifically, sampled pulse rate is used to derive at least one of several key breathing related parameters including but not limited to heartrate, heart rate variability, breathing rate, breathing rate variability, vagal tone, breath holding times. Personalized breathing course instructions follow regimens using PAP mask 200 to improve breathing functionality that may alleviate SDB symptoms. In FIG. 6, the sensor is built into the PAP mask 300 as sensor 302. One illustrative example of breath training is shown in FIG. 7 where the user starts with natural breathing at step 700 and proceeds through various steps of short breath holds, long breath holds, and soft breathing and finally finishing with natural breathing at step 702. During these breath training sessions, the user's physiological parameters (e.g. heart rate, blood oxygen level, heart-rate variability, breath rate, breath holding times, etc.) are monitored by the one or more sensors and compared to clinical data or user data from previous breath training exercises.

The breathing behavior data and their trends for the SDB patient are used daily or periodically to modify the PAP management data. This is provided either locally through a local wired or wireless connection or more usually via a secure internet connection as shown in FIG. 5 to databases 500 which may reside on a remote server device.

Modes' data, including changes in both Modes can be accessed securely by the modes' Health Care Provider, (HCP) 504 via a personal computer or smart device (e.g. phone, tablet, etc.). As an illustrative example, a patient suffering from SDB is provided with a PAP preprogrammed with an initial treatment protocol. The patient is also provided with a separate means, as described above, to undertake breath training with a regimen suggested by an HCP. This regimen may be based on a phenotype comparison.

Patient data collected separately from Modes I and II are tracked over time. As breath training improves breathing patterns, the management software from Mode II is modified and the apnea events monitored nightly. The combination of breath training and PAP data can be used to improve therapies in both modes. If and when nightly apnea events are reduced, the breath training regimens can be modified appropriately providing a virtual circle of combinatorial therapies.

A second embodiment is possible if the PAP device has its own inbuilt or attached computing device. This can replace the need completely or partly for a separate local computing device.

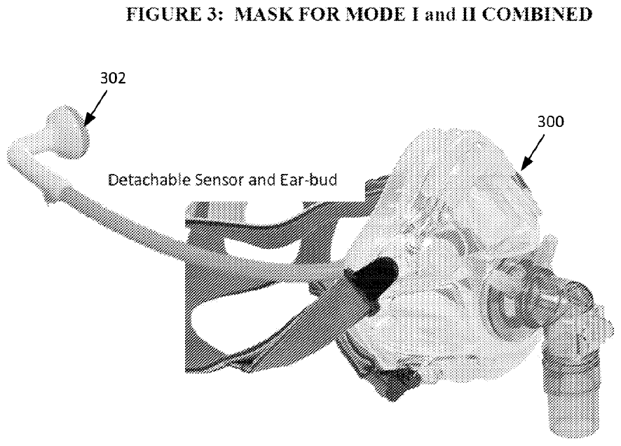

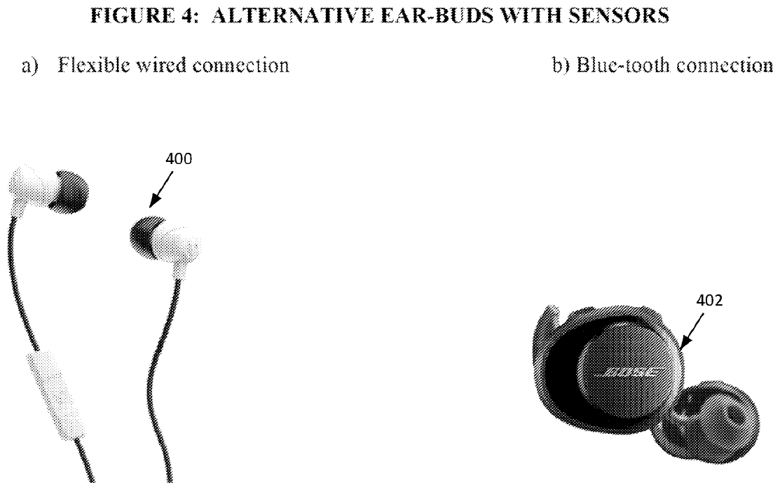

In one embodiment a detachable sensor extension can be employed to enable Mode I to be integrated with a Mode II device as shown in FIG. 3 where mask 300 is integrated with sensor 302 (e.g. heart rate sensor, blood oxygen saturation sensor, etc.). The extension may also serve as an audio output device (e.g. they may be headphones, earbuds, etc.). Connections may be rigid like earbud sensor 302 in FIG. 3, flexible like earbuds 400 in view (a) of FIG. 4 or wireless like earbuds 402 in view (b) of FIG. 4. These sensor extensions would be used in the wakened state for breath training. In this example, the integrated device can perform both PAP therapy and breath training exercises. The integrated device can modify the PAP therapy based on parameters measured during the breath training exercises or modify the breath training based on parameters measured during the PAP therapy.

The patient is supplied with a second apparatus that applies Positive Airway Pressure (PAP) during sleep to alleviate symptoms of SDB. A typical apparatus that operates in Mode II is shown in FIG. 2. The patient wears the mask during sleep and positive pressure is applied continuously or from time to time. Breathing patterns, such as apnea and hypopnea events are monitored and stored in an electronic memory often together with other physiological data. These data are called Mode II data. The data is provided from time to time to the patient's healthcare professional who may use it to modify the control algorithms stored in the apparatus. The apparatus for Mode II may be required for as long as the patient continues to have SDB symptoms.

The ability to exchange data between a device dedicated to Mode I breathe training, and a device dedicated to Mode II, PAP application provides advantages to the alleviation of SDB symptoms through greater convenience to the patient, enhanced information to the expert and improved therapeutic outcomes.

For example the patient uses a device in Mode I while awake according to a breath training protocol deemed appropriate stored in the device. The patient uses a second device in Mode II when sleeping and is subjected to one of the many PAP protocols. In Mode II, changes over time in SDB patterns are monitored and stored as part or all of Mode II data. Analysis of these data may be used to modify the breath training regimens stored in the first device for subsequent use in Mode I. As one example, as improvements are seen in the symptoms of SDB's over time by the Mode II device, the patient may be instructed to modify one or more of the intensity, length or frequency of breath training exercises guided by the Mode I device. As improvements are seen in the symptoms of SDB's over time by a Mode II apparatus, the patient may be advised that they can reduce the use of this device or even cease use of either one or both of the dedicated Mode I and II devices. In such a case, the patient may be instructed to periodically use the Mode II to determine whether their SDB symptoms have increased and whether they should again use either or both devices until the symptoms have been reduced to a level where they can again reduce or stop either or both modes.

In a second embodiment, the two functions, Mode I and Mode II operate within a single wearable device such as the device shown in FIG. 2. Such a device will have a mask means to provide PAP, means to monitor heart rate and breathing patterns during both sleep and wake periods, a data storage means, a data transfer means and a monaural or binaural means to provide the patient with audio inputs. The monaural or binaural ear-pieces may be a permanent part of the wearable device, or detachable providing greater comfort and convenience when operating in Mode II. Mode I operation is similar to that described above.

The patient uses the device in Mode I while awake according to a breath training protocol deemed appropriate stored in the device. The patient uses the device in Mode II when sleeping and is subjected to one of the many PAP protocols. In Mode II, changes over time in SDB patterns are monitored and stored. Analyses of these data may be used to modify the breath training regimens stored in the device for subsequent use in Mode I. For example, as improvements are seen in the symptoms of SDB's over time when in Mode II, the patient may be instructed to reduce one or more of the intensity, length or frequency of breath training exercises. As another example, as improvements are seen in the symptoms of SDB's over time in Mode II, the patient may be advised that they can reduce the use of the device in Mode II or even cease use in either or both of Mode I and II. In such a case, the patient may be instructed to periodically use the device in Mode II to determine whether their SDB symptoms have increased and whether they should again use either or both Modes I and II until the symptoms have been reduced to a level where they can again reduce or stop either or both modes.

Specifically breath training regimens used in Mode I may be modified depending on Mode II data in order to improve therapeutic procedures and provide enhanced outcome benefits to patients of SDB. Likewise, PAP therapy used in Mode II can be modified depending on Mode I data determined during the breath training regimens.

The following is a first detailed example demonstrating how algorithmically combining the control software for Mode I and Mode II can overcome existing problems with treatment methods and improve medical outcomes. In this example, the Mode II device may be manually or preferably automatically fully or partially de-activated by reducing the applied pressure to the patient's upper airways. If the patient is still suffering from OSA or CNSA, the device detects the times of the lengths (T^{II}1,2,3, . . . n) of a series of apnea events. From this data set the mean interrupt time T^{II}_{Average} and the maximum interrupt time T^{II}ₘₐₓ are calculated. These values are compared with the average breath holding time, T^{I}_{Average} achieved by the patient as recorded by a Mode I device. If T^{II}_{Average} is greater than T^{I}_{Average} then the target breath holding time in mode I is set as T^{II}_{Average}. A new breath training regimen designed to train the patient over a defined time period to reach the new target is then used for ongoing training. When T^{I}_{Average} exceeds T^{II}_{Average}, the Mode II device repeats the de-activation cycle and new values of T^{II}_{Average} and T^{II}ₘₐₓ are determined. The procedure is repeated until the current value of T^{II}ₘₐₓ has been exceeded by the current value of T^{I}_{Average}, the breath training regimen may be replaced or modified in each cycle. As the number and/or values of T^{II}1,2,3, . . . n decline, the Mode II device applied pressure levels can be decreased until de-activation exposes apnea events, at which time the feed-back cycles are repeated. In some cases, it may be possible to reduce the use or even cease use of the Mode II device. In addition, Mode I device use may also be reduced. In this case, Mode II may be periodically re-introduced and the full procedure repeated if necessary.

The following is a second example demonstrating how algorithmically, intimately combining the control software for Mode I and Mode II can overcome existing problems with treatment methods and improve medical outcomes. In this example, PAP control algorithms used in Mode II may be modified depending on Mode I data in order to improve therapeutic methods. If T^{I}_{Average} determined by the Mode I device is more than a factor X (e.g. percentage), typically 0.5 below T^{II}_{Average}, the Mode II device is programmed to reduce apnea events during sleep to a predetermined level of either or both AHI and ODI. Factor X is determined from time to time using data from both Mode I and Mode II devices. If factor X increases to a pre-determined value, for example 0.75, the Mode II device can be programmed to maintain AHI and/or ODI values at the same or lower levels. At the same time, or when X reaches another pre-determined value, for example, 0.8, the procedures in the first example are invoked.

In other examples, one of more of heart rate, heart rate variability, blood oxygen saturation, breath hold duration, among others may be monitored and compared to thresholds to determine trainee performance. These thresholds may be predetermined or may be based on the performance of previous training sessions.

In the case that a single apparatus is able to operate in both modes, data collected during operation in one mode many be used to initially define and/or subsequently modify the electronic control algorithms in the other mode and vice versa as described in the first example and the second example.

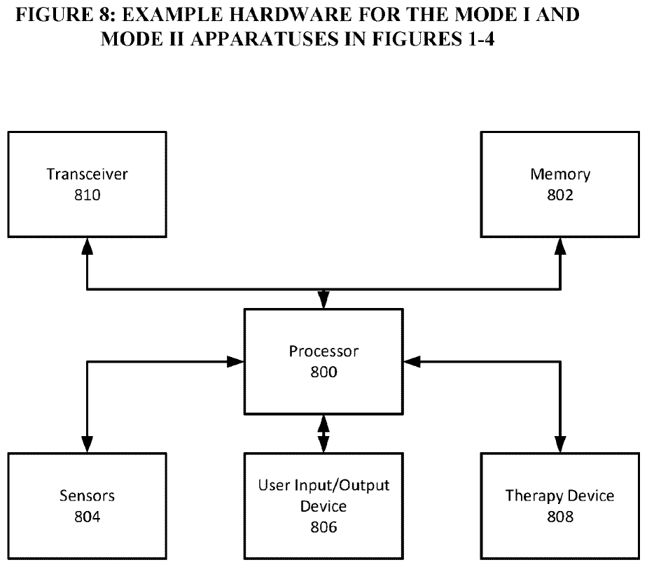

FIG. 8 shows example hardware for the Mode I and Mode II apparatuses shown in FIGS. 1-4. In general, the apparatuses in FIGS. 1-4 include one or more of processor 800 and memory 802 for executing the Mode I and Mode II algorithms, sensors 804 (e.g. heart rate sensor, blood oxygen saturation sensor, blood pressure sensor, air pressure sensor, etc.) for sensing Positive Airway Pressure interventions and physiological data, user input/output device 806 (e.g. buttons, display screen, etc.) for receiving input from the user and outputting data/feedback to the user, therapy device 808 (e.g. PAP machine) for applying therapy to the user and wireless transceiver 810 (e.g. WiFi, Bluetooth, Cellular, etc.) for communications between Mode I and Mode II devices and communications between these devices and external devices such as a server.

While the foregoing is directed to example embodiments described herein, other and further example embodiments may be devised without departing from the basic scope thereof. For example, aspects of the present disclosure may be implemented in hardware or software or a combination of hardware and software. One example embodiment described herein may be implemented as a program product for use with a computer system. The program(s) of the program product defines functions of the example embodiments (including the methods described herein) and may be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid-state random-access memory) on which alterable information is stored. Such computer-readable storage media, when carrying computer-readable instructions that direct the functions of the disclosed example embodiments, are example embodiments of the present disclosure.

It will be appreciated by those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A portable smart device designed to be held by a user or worn by the user, the portable smart device for determining a breathing quality of a user during a breath training session, the portable smart device comprising:
one or more sensors configured to detect physiological parameter values of the user during the breath training session; and
a processor coupled to the one or more sensors, the processor configured to:
receive Mode II physiological parameter values from a positive airway pressure (PAP) device utilized by the user during PAP therapy, the Mode II physiological parameter values measured by the PAP device during the PAP therapy,
develop and execute a breath training session based on the Mode II physiological parameter values,
analyze, the physiological parameter values of the user detected by the one or more sensors during the breath training session to determine Mode I physiological parameter values, the Mode I physiological parameter values measured by the portable smart device during the breath training session, and
transmit the physiological parameter values to the PAP device for adjustment of the PAP therapy.

2. The portable smart device of claim 1,
wherein the Mode II physiological parameter values include at least one of length and frequency of occurrence of Apnea events, and
wherein the Mode I physiological parameter values include at least one of heart rate, heart rate variability, breathing rate, breathing rate variability, vagal tone, breath holding capability, variations in breath holding capability, blood oxygen saturation level and blood oxygen saturation level variability.

3. The portable smart device of claim 1,
wherein the Mode II physiological parameter values include at least one of a mean duration and a maximum duration of Apnea events as measured during PAP therapy.

4. The portable smart device of claim 3,
wherein the processor is further configured to:
compare the at least one of the mean duration and the maximum duration of Apnea events to a Mode I breath holding target duration,
develop the breath training session by increasing the breath holding target duration in the breath training session when the mean duration and the maximum duration of Apnea events is greater than the Mode I breath holding target duration, and
transmit an adjustment parameter to the PAP device when the mean duration and the maximum duration of Apnea events is less than the Mode I breath holding target duration, the adjustment parameter instructing the PAP device to reduce pressure or shut off the PAP device for a least a duration of the PAP therapy.

5. The portable smart device of claim 1,
wherein the Mode II physiological parameter values are measured by deactivating or decreasing pressure for a duration of the PAP therapy.

6. A positive airway pressure (PAP) device having a mask worn by the user, the PAP device for monitoring apnea events during PAP therapy, the PAP device comprising:
a fan for applying positive airway pressure to the mask worn by the user during the PAP therapy;
one or more sensors configured to detect physiological parameter values of the user during PAP therapy; and
a processor coupled to the one or more sensors, the processor configured to:
receive Mode I physiological parameter values from a portable smart device, the Mode I physiological parameter values measured by the portable smart device during a breath training session,
develop and execute PAP therapy based on the Mode I physiological parameter values, analyze, the physiological parameter values of the user detected by the one or more sensors during the PAP therapy to determine Mode II physiological parameter values, and
transmit the Mode II physiological parameter values to the portable smart device for adjustment of the breath training.

7. The PAP device of claim 6,
wherein the Mode II physiological parameter values include at least one of length and frequency of occurrence of Apnea events, and
wherein the Mode I physiological parameter values include at least one of heart rate, heart rate variability, breathing rate, breathing rate variability, vagal tone, breath holding capability, variations in breath holding capability, blood oxygen saturation level and blood oxygen saturation level variability.

8. The PAP device of claim 6,
wherein the Mode II physiological parameter values include at least one of a mean duration and a maximum duration of Apnea events as measured during PAP therapy.

9. The PAP device of claim 8,
wherein the processor is further configured to:
develop the PAP therapy by increasing pressure during PAP therapy when the mean duration and the maximum duration of Apnea events is greater than a Mode I breath holding target duration, and
develop the PAP therapy by reducing pressure or shutting off the PAP device for a least a duration of the PAP therapy when the mean duration and the maximum duration of Apnea events is less than the Mode I breath holding target duration.

10. The PAP device of claim 6,
wherein the Mode II physiological parameter values are measured by deactivating or decreasing pressure for a duration of the PAP therapy.

11. An integrated positive airway pressure (PAP) device having a mask worn by the user, the PAP device for monitoring apnea events during PAP therapy, and for performing breath training, the PAP device comprising:
a fan for applying positive airway pressure to the mask worn by the user during the PAP therapy;
one or more sensors configured to detect physiological parameter values of the user during PAP therapy; and
a processor coupled to the one or more sensors, the processor configured to:
detect, via the one or more sensors, Mode I physiological parameter values measured by the PAP device during a breath training session,
develop and execute PAP therapy based on the Mode I physiological parameter values, analyze, the physiological parameter values of the user detected by the one or more sensors during the PAP therapy to determine Mode II physiological parameter values,
adjust the breath training breath training based on the Mode II physiological parameter values, and
adjust the PAP therapy based on the Mode I physiological parameter values.

12. The integrated PAP device of claim 11,
wherein the Mode II physiological parameter values include at least one of length and frequency of occurrence of Apnea events, and
wherein the Mode I physiological parameter values include at least one of heart rate, heart rate variability, breathing rate, breathing rate variability, vagal tone, breath holding capability, variations in breath holding capability, blood oxygen saturation level and blood oxygen saturation level variability.

13. The integrated PAP device of claim 11,
wherein the Mode II physiological parameter values include at least one of a mean duration and a maximum duration of Apnea events as measured during PAP therapy.

14. The integrated PAP device of claim 13,
wherein the processor is further configured to:
develop the PAP therapy by increasing pressure during PAP therapy when the mean duration and the maximum duration of Apnea events is greater than a Mode I breath holding target duration, and
develop the PAP therapy by reducing pressure or shutting off the PAP device for a least a duration of the PAP therapy when the mean duration and the maximum duration of Apnea events is less than the Mode I breath holding target duration.

15. The integrated PAP device of claim 11,
wherein the Mode II physiological parameter values are measured by deactivating or decreasing pressure for a duration of the PAP therapy.
